(19) United States
(12) Patent Application Publication
(10) Pub. No.: US 2008/0268538 A1
(43) Pub. Date: Oct. 30, 2008

(54) Device and Method to Prevent Culture Media Degradation

(76) Inventors: Robert Nordon, New South Wales (AU); Michael R. Doran, Red Deer (CA); Reinhold Deppisch, Hechingen (DE)

Correspondence Address: NIXON & VANDERHYE, PC, 901 NORTH GLEBE ROAD, 11TH FLOOR, ARLINGTON, VA, 22203, US

(21) Appl. No.: 11/662,374

(22) PCT Filed: Sep. 12, 2005
(86) PCT No.: PCT/AU2005/001384

§ 371 Date: May 2, 2008

(30) Foreign Application Priority Data
Sep. 10, 2004 (AU) 2004905188

(51) Int. Cl. C12N 5/06; C12M 1/00

(52) U.S. Cl. 435/388; 435/297.2

(57) ABSTRACT

The present invention provides a device for cell culture. The device comprises a culture zone, a selective sorption zone and culture media transfer means. Culture media is transferred or recirculated from the culture zone through the selective sorption zone which comprises at least one agent reactive with cytotoxic degradation products present in the culture media and back to the culture zone via the culture media transfer means.

### FIELD OF THE INVENTION

The present invention relates to a method and apparatus for cell culture.

### BACKGROUND OF THE INVENTION

One method of cell culture/expansion involves the use of a perfusion hollow fibre or porous flat substrates bioreactor. Hollow fibre bioreactors typically contain membranes that separate a first cell-containing compartment from a second compartment. The membrane may be a porous substrate or may be a barrier layer based substrate. In such a bioreactor, cell growth is typically in static suspension culture on the intercapillary side of hollow fibres in a module. Media is continually circulated through the extracapillary side of the hollow fibres in the module. This media provides essential oxygen and nutrients to the cells across the hollow fibre membrane.

Unfortunately culture media degrades at 37° C. and this degradation is accelerated in a circulating system which is required for the above method. Media containing glucose degrades at a rate dependent on several factors such as the sterilization process, pH, temperature and time.

One side effect of the degradation process is the formation of cytotoxic compounds which inhibit cell growth or even influence phenotypic change.

### SUMMARY OF THE INVENTION

In a first aspect the present invention provides a device for cell culture, the device comprising a culture zone, a selective sorption zone (e.g. a selective barrier layer in the membrane) and culture media transfer means, wherein culture media is transferred or recirculated from the culture zone through the selective sorption zone and back to the culture zone via the culture media transfer means and wherein the selective sorption zone comprises at least one agent, e.g. a chemical structure, entity or ligand reactive with cytotoxic degradation products present in the culture media. The agent may be present as a solute in the media or as a component of a solid phase bound binding site.

In a second aspect the present invention provides a cell culture vessel comprising a cell culture chamber and a selective sorption chamber positioned within the cell culture vessel, the selective sorption chamber comprising at least one agent reactive with cytotoxic degradation products present in the culture media and being formed such that the at least one agent reactive with cytotoxic degradation products is in contact with the culture media but prevented from contact with the cultured cells.

In a third aspect the present invention provides a method of culturing cells using the device or culture vessel of the first or second aspect of the present invention.

### BRIEF DESCRIPTION OF THE FIGURES

FIG. 1: Unmodified perfusion system without a selective sorption zone. Cells are grown in the cell expansion module and media is circulated in the direction of the arrows by a pump.

FIG. 2: Modified perfusion system with a selective sorption zone. Cells are grown in the cell expansion module and media is circulated in the direction of the arrows by a pump. The media detoxification module comprises the selective sorption zone.

FIG. 3: Shows an experimental rig.

FIG. 4: results of cytotoxicity bioassay of saline extracted after 1 day of recirculation in rigs A-D.

FIG. 5: results of cytotoxicity bioassay of saline extracted after 5 days of recirculation in rigs A-D.

FIG. 6: results of the bioassay of protein-free media extracted on day 1.

FIG. 7: results of the bioassay of protein-free media extracted on day 2.

FIG. 8: results of the bioassay of protein-free media extracted on day 3.

FIG. 9: results of the bioassay of protein-free media with albumin on extracapillary side. Media was extracted for bioassay on day 2.

FIG. 10: results of the bioassay of protein-free media with albumin on extracapillary side. Media was extracted for bioassay on day 3.

FIG. 11: Shows a cell culture vessel depicting different detoxification arrangements, i.e particulate or cellular components in separate compartement, sorbent or scavenger agents in a second compartment

### DETAILED DESCRIPTION

The present inventors have found that culture media is degraded by both chemical and physical means. Chemical degradation results in the production of reactive oxygen species e.g. dicarbonyl compounds which are derived from oxidative attack to glucose (Brash, Wieslander A, Linden T, Musi B, Speidel R, Beck W, Henle T, Deppisch R. Exogenous Uptake of Carbonyl Stress Promoting AGE Formation from Peritoneal Dialysis Fluids. Contrib Nephrol. Basel, Karger 2002, vol 131, pp 82-89.). Such breakdown products are cytotoxic to cells. Physical degradation occurs when proteins such as albumin are degraded by fluid shear stress or fluid/gas interfaces (foaming).

It has been shown that degradation products can be broken down, or inactivated, by several technical solutions. One solution includes the addition to the media of additives such as proteins which have high molecular weights such as albumin, lipid formulations or low molecular weight scavengers such as bisulfite, acetyl cysteine or metformin.

Other solutions may include the use of membranes such as polyflux (Gambro Co, Hechingen Germany) or other similar types of membranes which have intrinsic barrier properties. The permeability spectrum of the low flux (sieving coefficient below 5000 d) and high flux PAES-PVP or PAES-PA-PVP-membranes (<30.000), is important in this context, as the sieving coefficient for albumin is in both types <0.01% for high flux (for low flux <0.001%).

Precoating the membranes with scavenger molecules/agents like albumin, lipid formulations, other macromolecules or low molecular weight scavengers such as bisulfite, acetyl cysteine or metformin may also help to scavenge cytotoxic compounds.

While it has been found that albumin prevents cellular cytotoxicity by scavenging chemical degradation products, the ability of albumin to provide a sink for toxins is reduced as the physical effects of protein shear, which occurs during media recirculation, denature the albumin protein.

In a first aspect the present invention provides a device for cell culture, the device comprising a culture zone, a selective sorption zone and culture media transfer means, wherein culture media is transferred or recirculated from the culture zone through the selective sorption zone and back to the culture zone via the culture media transfer means and wherein the selective sorption zone comprises at least one agent reactive with cytotoxic degradation products present in the culture media.

In a second aspect the present invention provides a cell culture vessel comprising a cell culture chamber and a selective sorption chamber positioned within the cell culture vessel, the selective sorption chamber comprising at least one agent reactive with cytotoxic degradation products present in culture media and being formed such that the at least one agent reactive with cytotoxic degradation products is in contact with the culture media but prevented from contact with the cultured cells.

As used herein “selective sorption zone” is defined as a device or process that selectively removes cytotoxic products (of any origin, i.e. media degradation, polymer degradation, cell metabolism) without removing key culture media components. Selective sorption may be achieved by a range of adsorbents and physical configurations. The physical configuration of the “selective sorption zone” may be a hollow fibre membrane system used to dialyse the media against a molecule or suspended particle that selectively binds or neutralises media degradation products or a column that is filled with a resin that has the selective adsorption chemistry bound to a solid phase support.

In a preferred embodiment the agent and/or media supplement is selected from the group consisting of albumin, dicarbonyl scavengers (eg Penicillamine), antioxidants (eg glutathione), cultured cells, radical or general scavengers (eg ethylpyruvate or riboflavin), cells, enzymes and combinations thereof. It is preferred that the agent comprises albumin and mixtures with albumin.

In another preferred embodiment the recirculation of the agent is substantially limited to the selective sorption zone by the provision of a filtration/diffusion membrane or by solid phase immobilisation (eg attachment of agent/supplement to solid phase support). Where a filtration/diffusion membrane is provided, the agent may be substantially limited to the selective sorption zone by the pore size of the membrane having a molecular weight cut off that is lower than the molecular weight of the agent/supplement or by attaching the agent/supplement to a molecule with a molecular weight that is larger than the pore size of the membrane. Suitable molecules for attaching to the agent/supplement include hydrogel type of media (e.g. sepharose), porous beads (eg polystyrene beads), porous substrates (e.g. meltblown or woven materials), modified silica or nanoparticles or combinations.

In a further preferred embodiment cytotoxic products are substantially removed from the culture media without substantially removing non-cytotoxic products (eg essential and non-essential amino acids).

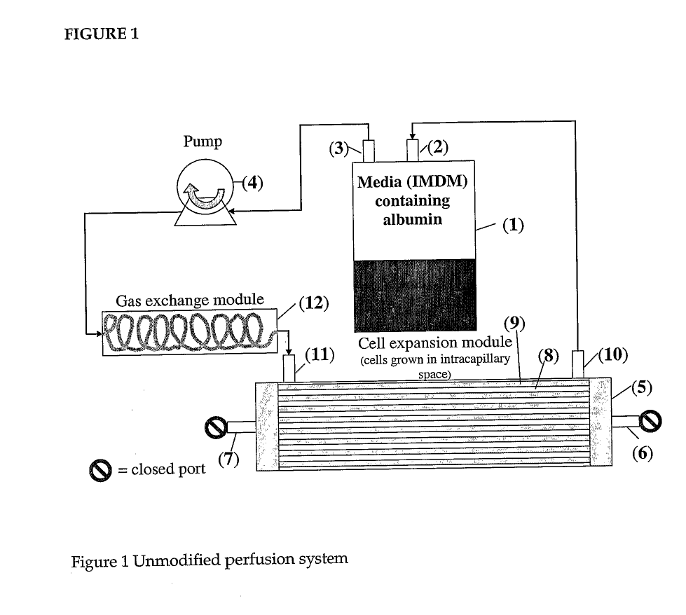

The proposed technical solution, i.e. utilizing a barrier or sink approach to scavenge unwanted by-products, may be further applied to solve another problem inherent in a bioreactor-type system. Polymer degradation is usually enhanced in oxidative or hydrolytic milieu. Upon breakdown of the polymer surface of the bioreactor rig, especially if the rigs are made of polyurethane materials, or has PVC softener molecules or other additives, the polymer degradation products may be scavenged by specific fluid or solid phase antibodies or adsorbents. FIG. 1 is a schematic view that illustrates an unmodified perfusion system. A media containment module (1) has ports (2) and (3) to allow a flow of media in and out of the media containment module (1). In FIG. 1 force generated by the pump (4) directs movement of the media in the direction of the arrows as indicated. Under force generated by the pump (4) media is directed from the media containment module (1) at port (3) through the gas exchange module (12) into the cell expansion module (5) at port (11).

The cell expansion module (5) has further ports (7) and (6) to permit the addition and extraction of media from the cell expansion module (5) as shown in FIG. 1. In operation of the perfusion system, the ports (6) and (7) may be closed to substantially prevent leakage of media from the system. The cell expansion module (5) has intracapillary (8) and extracapillary (9) spaces located therein. The extracapillary space (9) is substantially filled with media and cells may be grown in the intracapillary space (8) as shown in FIG. 1. Media pumped from the media containment module (1) enters the gas exchange module (12) and then enters the cell expansion module (5) through port (11). The media passes through the extracapillary space (9) of the cell expansion module (5) before exiting at port (10).

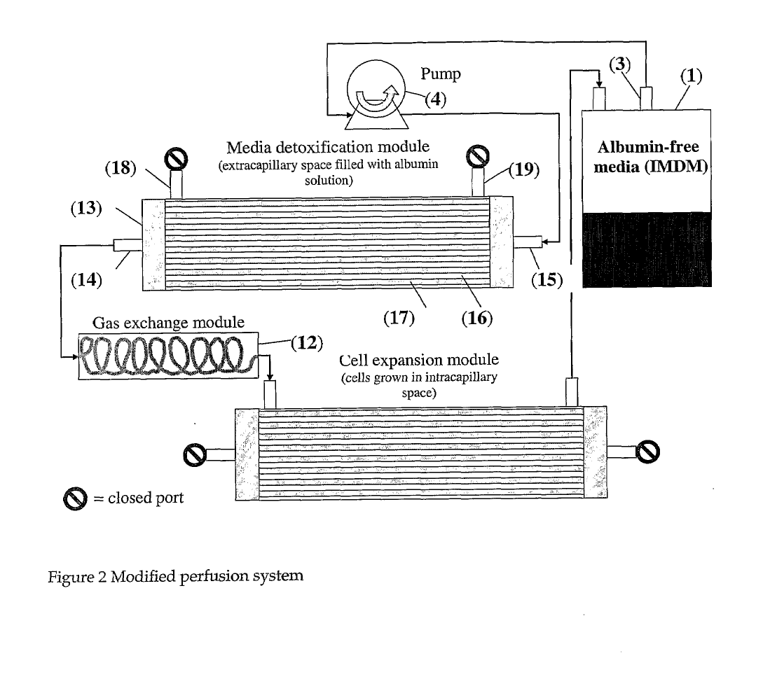

FIG. 2 is a schematic diagram that illustrates an embodiment of the present invention and depicts a modified perfusion system. In FIG. 2, the perfusion system as shown in FIG. 1 is modified by the addition of a media detoxification module (13) comprising ports (14) and (15), intracapillary (16) and extracapillary (17) spaces and sampling ports (18) and (19). In FIG. 2 force generated by the pump (4) directs movement of the media in the direction of the arrows as indicated. In the modified perfusion system as shown in FIG. 2, media exiting the media containment module (1) enters the media detoxification module (13) via port (15). The extracapillary space (17) comprises an agent/supplement (eg albumin). Media enters the media detoxification module (13) at port (15) and passes through the intracapillary space (16) of the media detoxification module (13). Cytotoxic degradation products present in the media are reactive with the agent/supplement in the extracapillary space (17) resulting in a reduction of the cytotoxic degradation products in the media exiting the media detoxification module (13) at port (14). Media exiting the media detoxification module at port (14) is directed through the gas exchange module (12) and into the cell expansion module ports (19) and (18) of the media detoxification module (13) permit the addition and extraction of agent/supplement from the media detoxification module (13) as shown in FIG. 2. In operation of the perfusion system, the sampling ports (18) and (19) may be closed to substantially prevent leakage of media from the system.

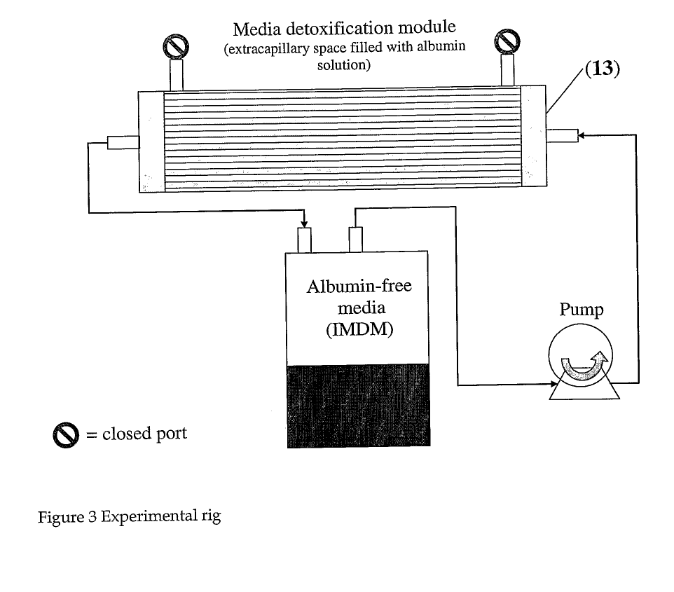

FIG. 3 is schematic diagram that illustrates an experimental rig. As shown in FIG. 3, the media detoxification module (13) may be comprised of either Pharmed tube alone (rig A), membrane consisting of alloy of polyarylethersulfone/PVP/polyamide (Gambro) (rig B), Baxter (Cellulose) (rig C) or AKZO (regenerated cellulose) (rig D) filtration diffusion modules.

In order that the nature of the present invention may be more clearly understood, preferred forms thereof will now be described with reference to the following non-limiting examples.

### EXAMPLE

The present inventors found that recirculated extracapillary media in the system shown in FIG. 1 degrades. The following experimental observations were made:
1. Media degrades at 37° C.
2. Media agents/supplements such as albumin, dicarbonyl scavengers (e.g. Penicillamine) and antioxidants (glutathione) help prevent media degradation
3. Media degradation is accelerated in a recirculating system (see FIG. 1), and is related to the rate of recirculation (extracapillary volume exchanges per unit time). Other factors which may accelerate the rate of media degradation include tubing length, polymer type, mode of sterilization and/or presence of air bubbles.
4. The rate of albumin degradation/precipitation is directly related to the rate of recirculation
5. For cell growth there is an absolute requirement for media supplementation with albumin (20 mg/ml).
6. Even though albumin will not cross the filtration/diffusion membrane, cells will not grow on the intracapillary side unless albumin is also on the extracapillary side of the membrane.

Based on these observations the following hypotheses were made:
1. Media is degraded by both chemical and physical means. Chemical degradation results in the production of reactive oxygen species (e.g. dicarbonyl compounds) that are cytotoxic. Proteins such as the albumin molecule are degraded by physical means such as fluid shear stress or fluid/gas interfaces (foaming).
2. Albumin prevents cellular cytotoxicity by scavenging chemical degradation products.
3. The ability of albumin to provide a sink for toxins is reduced as the shear effects of recirculation denature the albumin protein.
4. Membranes separating the cellular and the media compartments could act as a barrier if the scavenger molecule is above the sieving profile of the membrane itself or by containing binding sites for the toxins of interest.

### 1. System Modification to Prevent Shear-Induced Denaturation of Albumin:

The functional life of albumin could be increased if it were protected from shear-induced denaturation. On this premise we have designed a system where the recirculated extracapillary media does not contain albumin. In this system (shown in FIG. 2) the albumin is contained within the extracapillary side of a media detoxification module. The recirculated media passes through the intracapillary side of this module.

Our hypothesis is that cytotoxic degradation products will be removed from the recirculated media by the media detoxification module. The degradation products will diffuse across the selective sorption membrane and bind to albumin on the extracapillary side of the module. The functional lifespan of albumin will be lengthened because it is protected from shear-induced damage. The mass transfer of the described arrangement can be adapted by a number of specific means, i.e. flow, flow distribution, capillary diameter, etc.).

### 2. Experimental Design

To prove the principle that albumin could be isolated to a single unit of the system and still reduce media degradation while increasing protein life we designed the system shown in FIG. 3. This system recirculates protein free media through the intracapillary side of a media detoxification module. The extracapillary side of the module contains an albumin rich solution.

Four rigs were constructed (see FIG. 3). All used PharMed (HV-06485-14) tubing with a 1.6 mm inside diameter. Rig A has no module insert. Rig B has a Gambro Polyflux 6L membrane consisting of alloy of polyarylethersulfone/PVP/polyamide Capillary Dialyzer insert. Rig C has a Baxter CF-12 Capillary Dialyzer insert (Cuprophan). Rig D has an AKZO Cellulose Capillary Dialyzer insert (Cuprophan).

These configurations were chosen for the following reasons.
1. Rig A provides a control that will reveal the background media degradation associated with pump, tube set and reservoir vessel.
2. Rig B will allow us to assess the characteristics of membrane consisting of alloy of polyarylethersulfone/PVP/polyamide (Gambro) capillary material compared to Baxter's Cellulose capillary material used in Rig C. Both modules have an intracapillary surface area of around 1 m².
3. Rig D with the AKZO Module has 200 cm² of cellulose membrane. Thus the AKZO module will reveal if the 50 times greater surface area of the Baxter Module over the AKZO modifies the rate of degradation.

### 3. Biological Assay for Media Degradation

Media degradation is measured in this study using a biological assay (bioassay). The assay measures the inhibition of growth of a cell line (KGla) by degraded media (see below).

Media is Iscove's Modification of Dulbecco's Medium (IMDM) by ICN Biomedicals Inc. Media is dissolved in Baxter's Pyrogen free Water with 2 grams/l sodium bicarbonate and adjusted to pH 7.2.

Our bioassay is set up in the format shown in Table 1 by diluting recirculated media extract, saline or fresh media with fresh media containing 10% fetal calf serum (FCS).

The myeloid leukaemia cell line KGla is seeded in culture wells at cells per 2 ml culture and harvested 7 days later.

TABLE 1
Bioassay design

| | | | | | |
|---|---|---|---|---|---|
| 100% | 80% | 60% | 40% | 20% | 0% |

| | |
|---|---|
| Test wells | Media extract* |
| Negative control wells | Saline* |
| Positive control wells | Fresh media* |

*Diluted with fresh media containing 10% fetal calf serum

The negative control is saline diluted with increasing amounts of fresh media (with FCS). Dilution with saline determines whether growth inhibition could be related to depletion of nutrients rather than cytotoxicity. If cell growth is slower than negative controls it is assumed that cytotoxic chemicals are present in the media extract. The positive control is made up of fresh media (no FCS) diluted with increasing amounts of fresh media with FCS. This control represents the effect of protein on cell growth.

In three consecutive experiments, the recirculated fluid was saline, protein-free media without albumin in the extracapillary space of the module or recirculated protein-free media with albumin in the extracapillary space.

### 4. Saline Extraction

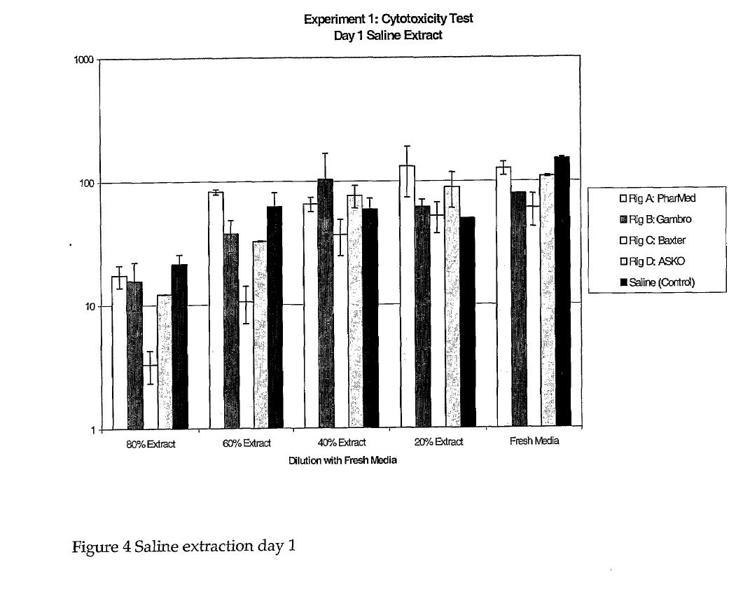

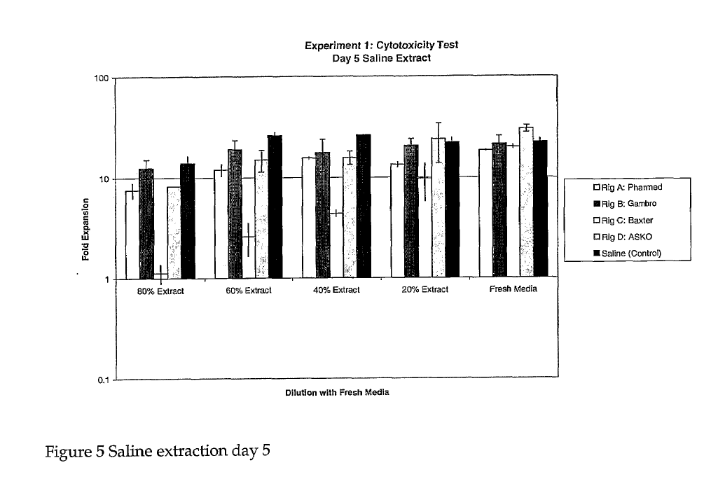

Rigs A-D were washed with normal saline before the saline extraction test. In each system 150 ml of saline is recirculated. The system is placed in a 37° C. 5% CO₂ incubator. The saline is recirculated at a flow rate of 1 ml/min. Samples of saline extract were taken after 1 day and 5 days of recirculation. The saline extract was tested for cytotoxicity using the bioassay. Results can be seen in FIG. 4 and FIG. 5.

There were similar bioassay results at day 1 and day 5 for all rigs and control with the exception of Rig C (Baxter module). Cell cultures performed in extract from the Baxter system had significantly lesser growth than the saline control. This indicates there is cytotoxicity associated with the Baxter CF-12 module. The toxic effects appear to be worse at day 5, consistent with leaching out of a toxic substance from the module.

### 5. Recirculation of Albumin-Free Media

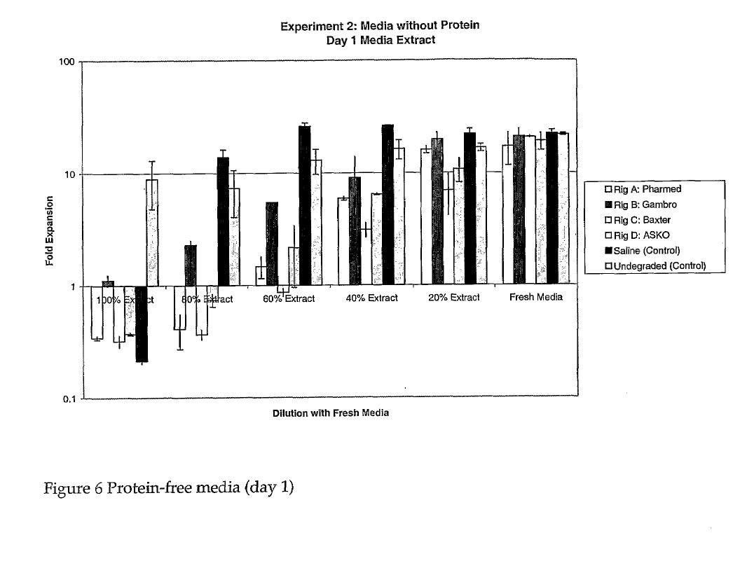

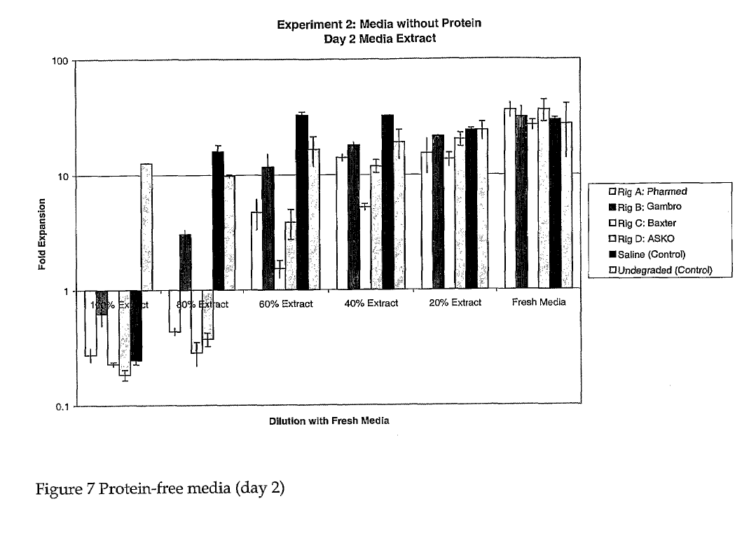

In this experiment IMDM without albumin is recirculated in each system for a period of three days. In each system 150 ml of media is recirculated. The extracapillary portion of the module is filled with media without albumin. The system is placed in a 37° C. 5% CO₂ incubator. The media is recirculated at a flow rate of 1 ml/min. Samples of media extract were taken after day 1, 2 and 3. FIG. 6, FIG. 7, and FIG. 8 show the results of the bioassay for media extracted on days 1, 2 and 3 respectively.

It is important to note that when fresh media alone is used, cell expansion is significant and nearly equal to fresh media containing 10% FCS. While there is no FCS in the media some is present in the cellular inoculum (˜1%). The level of growth for rigs A-D was lower than saline controls (all dilutions). Therefore toxic degradation products were present in all recirculated systems.

Rig C (Baxter module) had the lowest level of growth. The cytotoxicity is most likely related in part to direct cytotoxicity of the Baxter module (see saline extraction). In comparison to the other Rigs, Rig B (membrane consisting of alloy of polyarylethersulfone/PVP/polyamide (Gambro) module) had the lowest level of media degradation.

6. Recirculated Protein-Free Media with Albumin in the Extracapillary Space.

In this experiment we tested the hypothesis that albumin does not need to be dissolved in recirculated media to act as scavenger of degradation products. Degradation products can be absorbed across a selective sorption membrane by albumin. If this process does occur then it would be expected that media from such a system would perform well in the bioassay.

Albumin was dissolved in IMDM at the concentration shown in Table 3. These concentrations were calculated on the basis that the final concentration of albumin if distributed uniformly across both the intracapillary and extracapillary volumes would be 20 mg/ml, except for the AKZO module where the extracapillary volume was only 5 ml, and the distributed concentration was only 2 mg/ml. Protein-free media was recirculated on the intracapillary side of the module.

TABLE 3
Concentration of albumin in the extracapillary space.

| | | | | | Concentration | |
|---|---|---|---|---|---|---|
| | | | | | if dissolved | |
| | Volume of | | Albumin | | in EC and | |
| | EC space | | concentration | | IC media | |
| Rig B (Gambro) | 150 | ml | 40 | mg/ml | 20 | mg/ml |
| Rig C (Cuprophan large) | 50 | ml | 120 | mg/ml | 20 | mg/ml |
| Rig D (Cuprophan small) | 5 | ml | 120 | mg/ml | 2 | mg/ml |

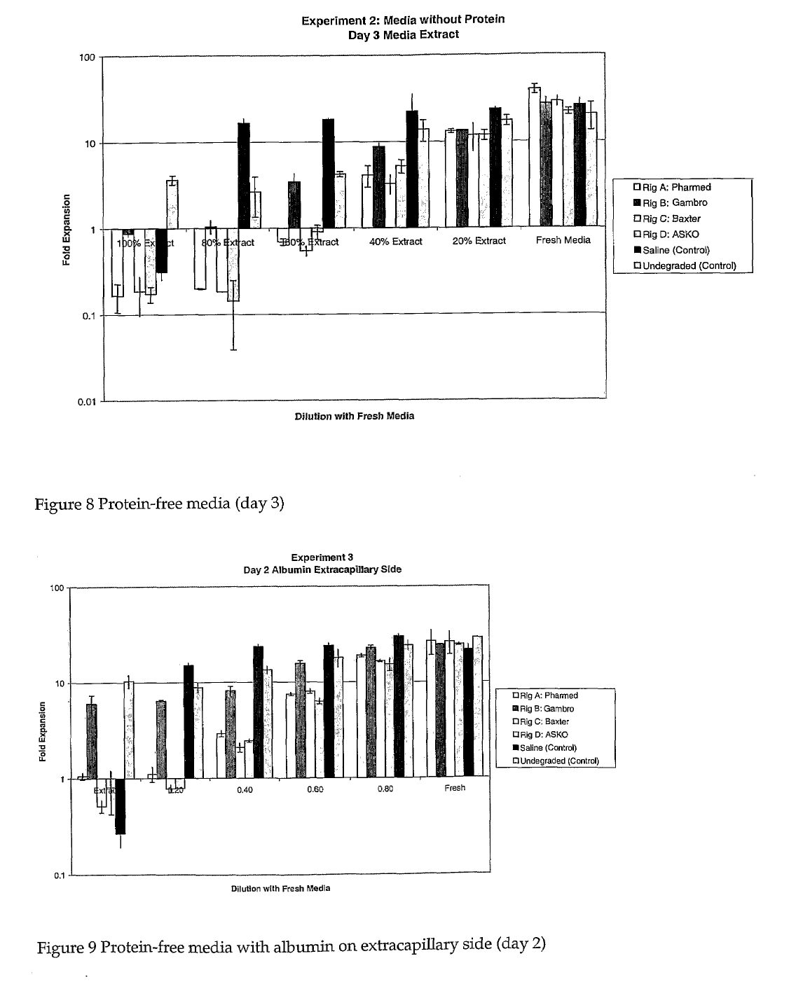

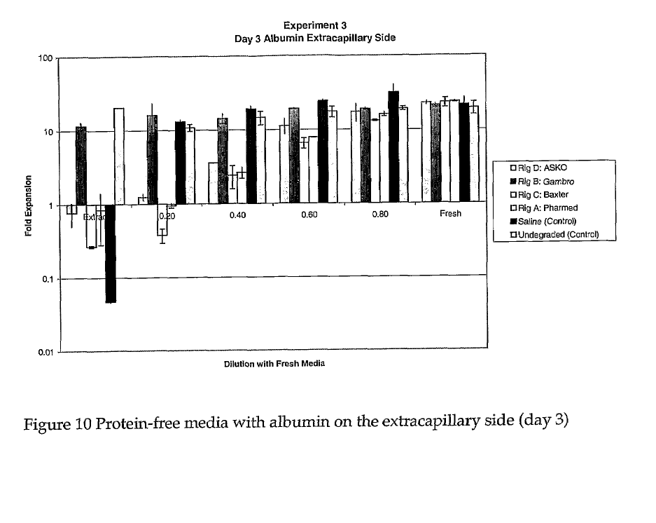

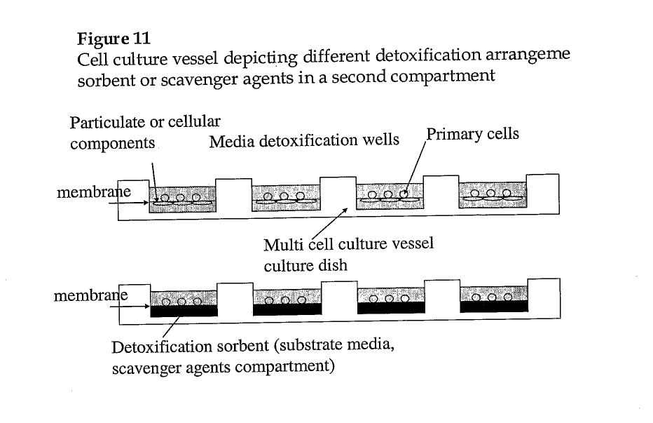

FIG. 9 and FIG. 10 show the results of the bioassay for protein-free media sampled on Days 2 and 3 respectively.

Consistent with experiments 1 and 2 Rig C containing the Baxter Module shows weaker growth. Rig A (Pharmed tube alone) and Rig D (ASKO module) had less growth than the saline control, indicating that they had some cytotoxicity. The membrane consisting of alloy of polyarylethersulfone/PVP/polyamide (Gambro) module (Rig B) has marginal or no media degradation when compared to the saline control. This result supports the hypothesis that albumin does not need to be dissolved in recirculated media to act as scavenger of degradation products. Degradation products can be absorbed across a selective sorption membrane by albumin.

### 7. Summary and Conclusion

Four experimental rigs were constructed as shown in FIG. 3. The control system consisted of only the tube sets, vessel and connectors (no media detoxification module). The three other rigs had inline membrane consisting of alloy of polyarylethersulfone/PVP/polyamide (Gambro), Baxter (Cellulose) or AKZO (Cellulose) filtration/diffusion (media detoxification) modules. Fluid passed through the intracapillary sides of these modules.

A current bioreactor design as per FIG. 1 includes albumin in the recirculated media. Accelerated media degradation and denaturation of albumin has been a significant problem limiting the utility of the bioreactor system.

The experimental results demonstrate that chemical media degradation can be reduced or prevented by selective removal of degradation products by a media detoxification module that contains albumin on the non-recirculated (extracapillary) side of the membrane.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

All publications mentioned in this specification are herein incorporated by reference. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia or elsewhere before the priority date of each claim of this application.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A device for cell culture, the device comprising a culture zone, a selective sorption zone and culture media transfer means, wherein culture media is transferred or recirculated from the culture zone through the selective sorption zone and back to the culture zone via the culture media transfer means and wherein the selective sorption zone comprises at least one agent reactive with cytotoxic degradation products present in the culture media.

2. A device according to claim 1 wherein the agent is selected from the group consisting of albumin, dicarbonyl scavengers (eg Penicillamine), antioxidants (eg glutathione), lipid formulations, cultured cells, radical or general scavengers (eg ethylpyruvate or riboflavin), cells, enzymes and combinations thereof.

3. A device according to claim 2 wherein the agent is albumin.

4. A device according to claim 1 wherein the agent is substantially limited to the selective sorption zone by the provision of a filtration/diffusion membrane or by solid phase immobilisation.

5. A device according to claim 1 wherein the agent is substantially limited to the selective sorption zone by the provision of a filtration/diffusion membrane in which the pore size of the membrane has a molecular weight cut off that is lower than the molecular weight of the agent or by attaching the agent to a molecule with a molecular weight that is larger than the pore size of the membrane.

6. A device according to claim 5 wherein the agent is attached to hydrogel type media (e.g., sepharose), porous beads (eg polystyrene beads), porous substrates (e.g. meltdown or woven materials), modified silica or nanoparticles or combinations.

7. A device according to claim 1 wherein the cytotoxic degradation products are substantially removed from the culture media without substantially removing non-cytotoxic products.

8. A cell culture vessel comprising a cell culture chamber and a selective sorption chamber positioned within the cell culture vessel, the selective sorption chamber comprising at least one agent reactive with cytotoxic degradation products present in culture media and being formed such that the at least one agent reactive with cytotoxic degradation products is in contact with the culture media but prevented from contact with the cultured cells.

9. A cell culture vessel according to claim 8 wherein the agent is selected from the group consisting of albumin, dicarbonyl scavengers (eg Penicillamine), antioxidants (eg glutathione), cultured cells, radical or general scavengers (eg ethylpyruvate or riboflavin), cells, enzymes and combinations thereof.

10. A cell culture vessel according to claim 9 wherein the agent is albumin.

11. A cell culture vessel according to claim 8 wherein the agent is substantially limited to the selective sorption zone by the provision of a filtration/diffusion membrane or by solid phase immobilisation.

12. A cell culture vessel according to claim 8 wherein the agent is substantially limited to the selective sorption zone by the provision of a filtration/diffusion membrane in which the pore size of the membrane has a molecular weight cut off that is lower than the molecular weight of the agent or by attaching the agent to a molecule with a molecular weight that is larger than the pore size of the membrane.

13. A cell culture vessel according to claim 12 wherein the agent is attached to hydrogel type media (e.g. sepharose), porous beads (eg polystyrene beads), porous substrates (e.g. meltblown or woven materials), modified silica or nanoparticles or combinations.

14. A cell culture vessel according to claim 8 wherein the cytotoxic degradation products are substantially removed from the culture media without substantially removing non-cytotoxic products.

15. A method of culturing cells using the device according to claim 1 or the culture vessel as defined above.
